(19) United States
(12) Patent Application Publication
(10) Pub. No.: US 2026/0074030 A1
(43) Pub. Date: Mar. 12, 2026

(54) PROGNOSTIC TOOLS FOR CLINICAL TRIAL ENROLLMENT

(71) Applicant: PERCEIV RESEARCH INC., Montreal (CA)

(72) Inventors: Christian DANSEREAU, Montreal (CA); Angela TAM, Montreal (CA); Cesar LAURENT, Montreal (CA)

(21) Appl. No.: 19/325,779

(22) Filed: Sep. 11, 2025

Related U.S. Application Data
(60) Provisional application No. 63/694,116, filed on Sep. 12, 2024.

(51) Int. Cl. G16H 10/20; G16H 50/20

(52) U.S. Cl. CPC G16H 10/20; G16H 50/20

(57) ABSTRACT

Methods and systems for using prognostic model(s) during a clinical trial enrollment process are disclosed. The method comprises acquiring a trial dataset comprising individual-specific data for a pool of trial individuals; and determining, using a pre-trained model an enrollable set of trial individuals from the pool of trial individuals, wherein: the pre-trained model is a prognostic model that has been trained to forecast a likelihood of progression of a disease or a condition after a pre-determined interval; and the pre-trained model has a dynamic operating point.

### CROSS-REFERENCE TO RELATED APPLICATION

This application claims the benefit of U.S. Provisional Patent Application No. 63/694,116, filed Sep. 12, 2024, which is incorporated by reference herein in its entirety.

### FIELD

The present technology relates generally to clinical trial enrollment, and more specifically, to prognostic tools for determining individuals to enroll in a clinical trial.

### BACKGROUND

Clinical trials are systematic investigations aimed at evaluating the efficacy and safety of new treatments, such as pharmaceutical drugs, in human subjects. These trials are essential for developing new therapies and ensuring their safety and effectiveness for patients.

A clinical trial may begin with trial design, where the objectives of the trial are defined, a detailed study protocol is developed, and necessary approvals are obtained from regulatory bodies and ethics committees. During patient recruitment, enrichment strategies may be implemented to select a patient population in which a drug effect, if present, is more likely to be observable. For example, practical enrichment may involve selecting patients that do not have other comorbid conditions that might obscure the drug's effect thereby reducing noise and heterogeneity in the trial data. During a clinical trial, different study frameworks may be employed such as, for example, randomization of patients to treatment and control groups to mitigate bias, maintenance of blinding to prevent bias in assessing outcomes, and systematic data collection according to the study protocol. In the data analysis phase, statistical methods can be used to determine the treatment's effectiveness, and interim analyses may be performed to check for early evidence of benefit or harm. Post-trial activities may involve compiling results, presenting findings in scientific meetings, publishing them in peer-reviewed journals, and submitting data to regulatory agencies for drug approval if the results are positive. Ongoing monitoring of the drug's safety and effectiveness may continue in a broader population after approval.

By incorporating enrichment strategies into the design and execution of clinical trials, researchers can enhance the likelihood of detecting true drug effects, thus improving the trial's efficiency and increasing the probability of successful outcomes. Current enrichment strategies include the use of biomarkers to stage and enrich the population investigated. Prognostic and predictive enrichment may be used to further refine this process.

Broadly speaking, prognostic enrichment focuses on enrolling patients more likely to experience the event of interest, such as progression of a disease or a condition.

A challenge with enrichment strategies is the tension between having a low enough inclusion criteria threshold so as not exclude potentially valuable individuals for the trial versus including a too heterogeneous population in terms of their natural outcome which can lead to more “noise” in the data and losing statistical power. While selecting a very homogenous sample with highly restrictive inclusion criteria may allow for better enrichment, and consequently greater statistical power, the cost and time for patient enrollment are often significantly increased, resulting in delays in the start and completion of trials.

Therefore, there is a need for more efficient enrichment strategies and enrollment processes for clinical trials.

### SUMMARY

Developers have devised methods and processors for overcoming at least some drawbacks present in prior art solutions.

In at least some embodiments of the present technology, there are provided methods and systems used in the context of prognostic enrichment strategies for clinical trials. Implementing predictive enrichment strategies as disclosed herein may allow faster development and approval of new therapies for targeted populations.

Developers of the present technology have realized that variability in progression of a disease or a condition may lead to more trial failures, and larger and/or longer trials. In some cases, biotech and/or pharmaceutical companies may need to compensate with larger and longer trials that are more expensive. In other words, it is desirable to predict how patients will progress without treatment to select an optimal population who is likely to show benefit in the time window of the trial. In one non-limiting example, Alzheimer's is a disease in which some patients may stay stable over many years, whereas other individuals may decline at various rates. In this non-limiting example, developers have realized that it may be difficult to assess, at a given moment in time (e.g., at the time of screening patients for the clinical trial), whether a given patient will be stable or will decline.

Developers of the present technology have realized that, for at least some diseases and conditions, it is desirable to select an optimal population of patients in terms of clinical trial enrollment, such as a more homogenous population rather than a larger heterogeneous population. It should be noted that populations with heterogeneous progression may create “variability” in the data, making it more difficult to obtain statistically significant findings in terms of the efficacy of the treatment being tested.

In some aspects of the present technology, there is provided an AI-driven prognostic method and system for forecasting progression of a disease or condition. The prognostic method and system may be used during clinical trial enrollment. In at least some implementations of the present technology, the prognostic method and system may be used to forecast progression of age-related disorders. In one implementation, the prognostic method and system may be used to forecast progression of cognitive decline in populations with Alzheimer's disease or any other neurodegenerative condition.

In some aspects of the present technology, there is provided a method and a system for trial enrollment comprising use of an AI-driven prognostic model which has been trained to take into account a progression of the disease or condition in the individual. The prognostic model may utilize dynamic inclusion and/or exclusion criteria for determining individuals to include in the clinical trial.

Embodiments of the present technology can be used for enrollment in trials evaluating treatment of any disease or condition, such as neurodegenerative conditions, e.g. Alzheimer's disease, Parkinson, all-cause dementia, etc.

In certain embodiments, advantageously, a reduction in screen failure rate is observed compared to prior art methods of screening for trials such as those that rely on the presence of a biomarker alone. By screen failure rate is meant the proportion of participants who are screened but ultimately do not meet the inclusion criteria for the clinical trial out of all screened participants.

In some embodiments of the present technology, methods and systems disclosed herein may be used to comparatively reduce screen failure rates for a same enrolled population sample size than conventional techniques. In other embodiments of the present technology, methods and systems disclosed herein may be used to comparatively reduce an enrolled population sample size for a same screen failure rate than conventional techniques.

In at least some embodiments of the present technology, there is provided a machine learning model trained to forecast progression of a disease or a condition in a given individual. The trained machine learning model is associated with a plurality of operating points for adjusting a performance of the model based on a selection criteria.

It is contemplated that using the pre-trained prognostic model as disclosed herein may allow at least one of: accelerating a clinical trial without compromising on sample quality, reducing screen failure rate of a clinical trial using prognostic information, removing a specific cutoff or range on a biomarker value during an enrollment processed in a clinical trial, and reducing the number of individuals to screen during a clinical trial's enrollment phase using a predictive model.

In a first broad aspect of the present technology, there is provided a method for an enrollment selection process in a clinical trial, the method comprising: during a calibration phase of a pre-trained prognostic model: acquiring a calibration dataset comprising individual-specific data for a pool of calibration individuals, the pool of calibration individuals matching requirements of the clinical trial; determining a reference set of calibration individuals from the pool of calibration individuals that are to be enrolled based on a biomarker-driven selection process applied onto the individual-specific data; determining a reference sample size requirement and a reference screen failure rate for the reference set of calibration individuals; for each of a plurality of candidate operating points of the pre-trained prognostic model: determining, using the pre-trained prognostic model with a given candidate operating point, a given set of calibration individuals from the pool of calibration individuals that are to be enrolled based on a model-driven selection process, the given candidate operating point being a candidate classification threshold for the pre-trained prognostic model for classifying a given calibration individual as being part of an enrolled class and a rejected class; determining a given sample size requirement and a given screen failure rate for the given set of calibration individuals determined using the given candidate operation point; determining a target operating point for the pre-trained prognostic model amongst the plurality of candidate operating points based on a comparison between at least one of: the reference sample size requirement and respective sample size requirements of the plurality of candidate operating points; and the reference screen failure rate and respective screen failure rates of the plurality of candidate operating points; during the enrollment phase of the clinical trial: acquiring a trial dataset comprising individual-specific data for a pool of trial individuals; determining, using the prognostic model with the target operating point, an enrollable set of trial individuals from the pool of trial individuals.

In some embodiments of the method, the target operating point is an upper target operating point determined based on a comparison of the reference sample size requirement and the respective sample size requirements of the plurality of candidate operating points, the upper target operating point for reducing a screen failure rate of the enrolled set of trial individuals for a same sample size requirement of the enrolled set of trial individuals if the biomarker-driven process has been used to determine the enrolled set of trial individuals.

In some embodiments of the method, the target operating point is a lower target operating point determined based on a comparison of the reference screen failure rate and the respective screen failure rates of the plurality of candidate operating points, the upper target operating point for reducing a sample size requirement of the enrolled set of trial individual for a same screen failure rate of the enrolled set of trial individuals if the biomarker-driven process has been used to determine the enrolled set of trial individuals.

In some embodiments of the method, the target operating point is a range of target operating points determined based on both (i) the reference sample size requirement and the respective sample size requirements of the plurality of candidate operating points, and (ii) the reference screen failure rate and the respective screen failure rates of the plurality of candidate operating points, and wherein the determining the enrolled set of trial individuals comprises using the prognostic model with at least one of operating point within the range of target operating points.

In some embodiments of the method, the individual-specific data comprises multimodal data.

In some embodiments of the method, the individual-specific data comprises biomarker data.

In some embodiments of the method, the pre-trained prognostic model has been trained to forecast a likelihood of progression of a disease or a condition after a pre-determined interval.

In some embodiments of the method, the likelihood is a binary value.

In some embodiments of the method, the likelihood is a spectrum of values.

In some embodiments of the method, the pre-trained prognostic model is at least one of: a bayesian model, a support vector machine, a linear regression model, a random forest model, a deep learning model, an ensemble-based model.

In some embodiments of the method, the method further comprises storing the determined enrollable set of individuals in a memory.

In some embodiments of the method, the method further comprises causing an enrollment of the enrollable set of individuals in the trial.

In a second broad aspect of the present technology, there is provided a system for an enrollment selection process in a clinical trial, the system being configured to: during a calibration phase of a pre-trained prognostic model: acquire a calibration dataset comprising individual-specific data for a pool of calibration individuals, the pool of calibration individuals matching requirements of the clinical trial; determine a reference set of calibration individuals from the pool of calibration individuals that are to be enrolled based on a biomarker-driven selection process applied onto the individual-specific data; determine a reference sample size requirement and a reference screen failure rate for the reference set of calibration individuals; for each of a plurality of candidate operating points of the pre-trained prognostic model: determine, using the pre-trained prognostic model with a given candidate operating point, a given set of calibration individuals from the pool of calibration individuals that are to be enrolled based on a model-driven selection process, the given candidate operating point being a candidate classification threshold for the pre-trained prognostic model for classifying a given calibration individual as being part of an enrolled class and a rejected class; determine a given sample size requirement and a given screen failure rate for the given set of calibration individuals determined using the given candidate operation point; determine a target operating point for the pre-trained prognostic model amongst the plurality of candidate operating points based on a comparison between at least one of: the reference sample size requirement and respective sample size requirements of the plurality of candidate operating points; and the reference screen failure rate and respective screen failure rates of the plurality of candidate operating points; during the enrollment phase of the clinical trial: acquire a trial dataset comprising individual-specific data for a pool of trial individuals; determine, using the prognostic model with the target operating point, an enrolled set of trial individuals from the pool of trial individuals.

In some embodiments of the server system, the target operating point is an upper target operating point determined based on a comparison of the reference sample size requirement and the respective sample size requirements of the plurality of candidate operating points, the upper target operating point for reducing a screen failure rate of the enrolled set of trial individuals for a same sample size requirement of the enrolled set of trial individuals if the biomarker-driven process has been used to determine the enrolled set of trial individuals.

In some embodiments of the server system, the target operating point is a lower target operating point determined based on a comparison of the reference failure rate and the respective failure rates of the plurality of candidate operating points, the upper target operating point for reducing a sample size requirement of the enrolled set of trial individual for a same screen failure rate of the enrolled set of trial individuals if the biomarker-driven process has been used to determine the enrolled set of trial individuals.

In some embodiments of the server system, the target operating point is a range of target operating points determined based on both (i) the reference sample size requirement and the respective sample size requirements of the plurality of candidate operating points, and (ii) the reference screen failure rate and the respective screen failure rates of the plurality of candidate operating points, and wherein the determining the enrolled set of trial individuals comprises using the prognostic model with at least one of operating point within the range of target operating points.

In some embodiments of the server system, the individual-specific data comprises multimodal data.

In some embodiments of the server system, the individual-specific data comprises biomarker data.

In some embodiments of the server system, the pre-trained prognostic model has been trained to forecast a likelihood of progression of a disease or a condition after a pre-determined interval.

In some embodiments of the server system, the likelihood is a binary value.

In some embodiments of the server system, the likelihood is a spectrum of values.

In some embodiments of the server system, the pre-trained prognostic model is at least one of: a bayesian model, a support vector machine, a linear regression model, a random forest model, a deep learning model, an ensemble-based model.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a schematic illustration of a computer system, in accordance with at least some non-limiting embodiments of the present technology.

FIG. 2 is a schematic illustration of a networked system, in accordance with at least some non-limiting embodiments of the present technology.

FIG. 3A is a schematic illustration of the conventional enrollment selection process.

FIGS. 3B and 3C are schematic illustrations of different scenarios of the conventional enrollment selection process of FIG. 3A using biomarker analysis.

FIG. 4 is a schematic illustration of an enrollment selection process with a prognostic model, in accordance with at least some non-limiting embodiments of the present technology.

FIG. 5 is a schematic illustration of a population subjected to the enrollment selection process of FIG. 4.

FIG. 6 is a schematic illustration of a training iteration of the prognostic model of FIG. 4.

FIG. 7 is a scheme-block illustration of a method for determining a reference value for a biomarker-driven process, in accordance with at least some non-limiting embodiments of the present technology.

FIG. 8 is a scheme-block illustration of a method of determining performance of the prognostic model for a plurality of candidate operating points, in accordance with at least some non-limiting embodiments of the present technology.

FIG. 9 is a graphical representation of performance of the prognostic model in accordance with at least some non-limiting embodiments of the present technology.

FIG. 10 is a scheme-block illustration of a method executable by a server of FIG. 2, in accordance with at least some non-limiting embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

In the context of the present specification, a “server” is a computer program that is running on appropriate hardware and is capable of receiving requests (e.g., from client devices) over a network, and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expression “at least one server”.

In the context of the present specification, “client device” is any computer hardware that is capable of running software appropriate to the relevant task at hand. Thus, some (non-limiting) examples of client devices include personal computers (desktops, laptops, netbooks, etc.), smartphones, and tablets, as well as network equipment such as routers, switches, and gateways. It should be noted that a device acting as a client device in the present context is not precluded from acting as a server to other client devices. The use of the expression “a client device” does not preclude multiple client devices being used in receiving/sending, carrying out or causing to be carried out any task or request, or the consequences of any task or request, or steps of any method described herein.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, the expression “component” is meant to include software (appropriate to a particular hardware context) that is both necessary and sufficient to achieve the specific function(s) being referenced.

In the context of the present specification, the expression “computer usable information storage medium” is intended to include media of any nature and kind whatsoever, including RAM, ROM, disks (CD-ROMs, DVDs, floppy disks, hard drivers, etc.), USB keys, solid state-drives, tape drives, etc.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

### Computer System

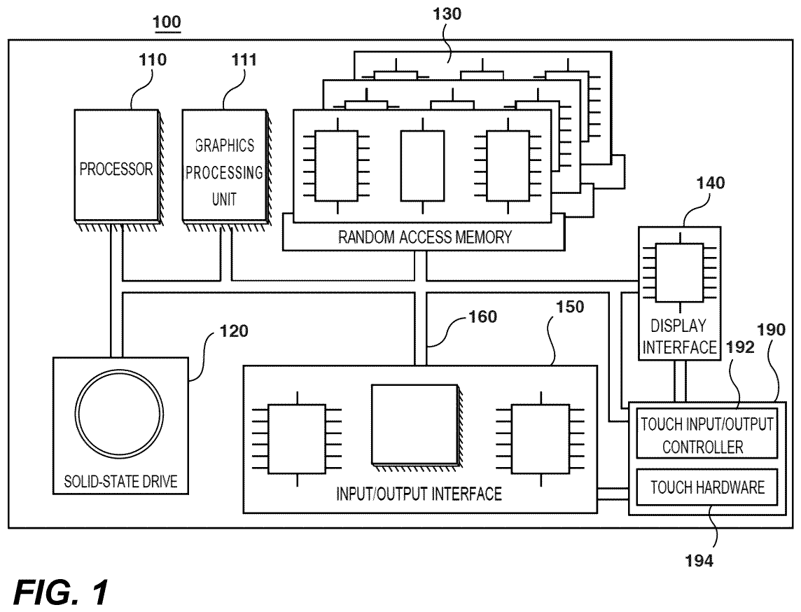

With reference to FIG. 1, there is depicted a computer system 100 suitable for use with some implementations of the present technology. The computer system 100 comprises various hardware components including one or more single or multi-core processors collectively represented by a processor 110, a graphics processing unit (GPU) 111, a solid-state drive 120, a random-access memory 130, a display interface 140, and an input/output interface 150.

According to implementations of the present technology, the solid-state drive 120 stores program instructions suitable for being loaded into the random-access memory 130 and executed by the processor 110 and/or the GPU 111. For example, the program instructions may be part of a library and/or an application.

Communication between the various components of the computer system 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. It is noted that some components of the computer system 100 can be omitted in some non-limiting embodiments of the present technology. For example, the keyboard and the mouse (both not separately depicted) can be omitted, especially (but not limited to) where the computer system 100 is implemented as a compact electronic device.

Broadly speaking, the touchscreen 190 may comprise touch hardware 194 and a touch input/output controller 192 allowing communication with the display interface 140 and/or the one or more internal and/or external buses 160. In some embodiments, the touch hardware 194 may comprise pressure-sensitive cells embedded in a layer of a display allowing detection of a physical interaction between a user and the display.

It should be noted that various implementations of the computer system 100 are contemplated. As it will become apparent from the description herein further below, one or more computer systems connected over a communication network may be implemented similarly to the computer system 100, without departing from the scope of the present technology.

### Networked Environment

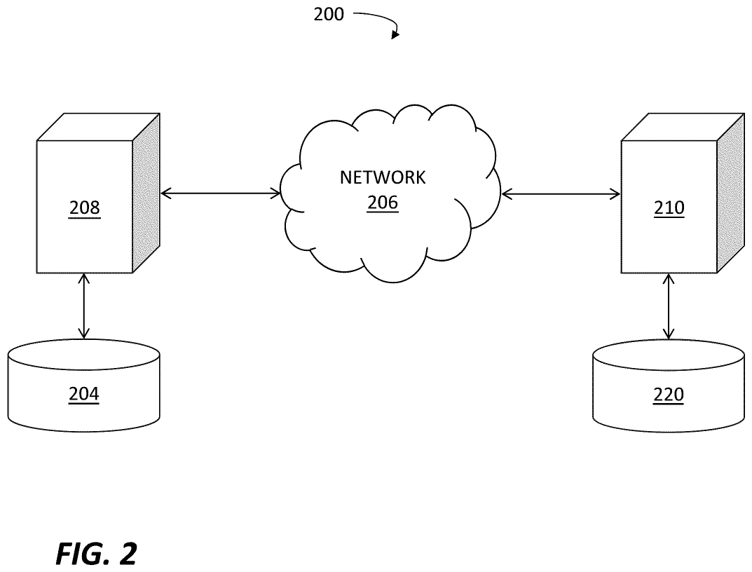

Referring to FIG. 2, there is shown a schematic diagram of a system 200, the system 200 being suitable for implementing non-limiting embodiments of the present technology. It is to be expressly understood that the system 200 as depicted is merely an illustrative implementation of the present technology. Thus, the description thereof that follows is intended to be only a description of illustrative examples of the present technology. Broadly speaking, the system 200 is configured to transmit data associated with a clinical trial.

The system 200 comprises the communication network 206. In one non-limiting example, the communication network 206 may be implemented as the Internet. In other non-limiting examples, the communication network 206 may be implemented differently, such as any wide-area communication network, local-area communication network, a private communication network and the like. In fact, how the communication network 206 is implemented is not limiting and will depend on inter alia how other components of the system 200 are implemented.

The purpose of the communication network 206 is to communicatively couple at least some of the components of the system 200 a resource server 208 and a system server 210. For example, this means that the resource server 208 is accessible via the communication network 206 by the system server 210.

The communication network 206 may be used to transmit data packets between the resource server 208 and the system server 210. For example, the communication network 206 may be used to transmit data requests from the system server 210 to the resource server 208, and vice versa.

The resource server 208 may be implemented as a conventional computer server. In a non-limiting example of an embodiment of the present technology, a given one of the resource server 208 may be implemented as a Dell™ PowerEdge™ Server running the Microsoft™ Windows Server™ operating system. The resource server 208 may also be implemented in any other suitable hardware and/or software and/or firmware or a combination thereof. Although in FIG. 2 a single resource server is illustrated, it should be understood that the resource server 208 may be embodied as a plurality of resource servers implemented via single or different operators, without departing from the scope of the present technology.

The resource server 208 is configured to host data about candidate populations for clinical trials. Which type of resources the resource server 208 is hosting is not limiting. However, in some embodiments of the present technology, the resources may comprise digital content such as text files, image files, audio files, video files, and the like. The resource server 208 may be accessed by the system server 210 in order to retrieve requested data stored on the resource server 208.

The system 200 comprises the system server 210 that may be implemented as a conventional computer server. Needless to say, the system server 210 may be implemented in any suitable hardware and/or software and/or firmware or a combination thereof. In the depicted non-limiting embodiment of present technology, the system server 210 is a single server. In alternative non-limiting embodiments of the present technology, the functionality of the system server 210 may be distributed and may be implemented via multiple servers.

Generally speaking, the system server 210 is under control and/or management of a prognostic system provider such as, for example, an operator of the prognostic system. As such, the system server 210 may be configured to host one or more components of the prognostic system to process data for the purpose of aiding in a clinical trial process.

For example, the system server 210 may receive the data from the resource server 208 about a plurality of individuals (e.g., anonymized data) and one or more clinical trial requirements. The system server 210 may process the data provided from the resource server 208 and return processing outcomes to the resource server 208.

The processing outcomes may take many forms. However, in one non-limiting example of the present technology, the processing outcomes may be a group of selected candidates for the clinical trial, and a group of non-selected candidates for the clinical trial. The system server 210 may be configured to determine which candidates are to be enrolled in the clinical trials and which are not. The results of this decision-making process can be securely transmitted back to the resource server 208 via the network 206. How the system server 210 is configured to generate the processing outcomes will be discussed below.

A database system 204 is configured to store data associated with a clinical trial. For example, the database system 204 may store trial-specific information and candidate-specific information, such as personal details, medical history, and other relevant data points. A database system 220 is configured to store data and modules for supporting generation of processing outcomes by the system server 210. For example, the database system 220 may store training data for training one or more machine learning models.

It is contemplated that the system 200 may be configured to provide a flow of information between the resource server 208 and the system server 210 and efficient processing of candidate data. It is contemplated that data exchanged between the resource server 208 and the system server 210 may be anonymized and/or encrypted to maintain confidentiality and integrity of data. To that end, both servers may implement strict access control mechanisms to prevent unauthorized access to sensitive information.

### Clinical Trial & Enrollment

It should be noted that a clinical trial may involve a plurality of steps.

An initial step involves the identification of potential candidates by reviewing medical records to confirm a diagnosis and determine the stage of a disease or a condition. Hospitals and clinics often utilize electronic health records (EHRs) to filter patients based on these criteria. This helps in narrowing down the pool to those who are experiencing a specific phase of the disease or the condition relevant to the study.

Once potential candidates are identified, the next step is pre-screening these individuals based on specific inclusion criteria outlined in the trial protocol. These criteria might include age, sex, and other relevant demographic or health-related factors. Pre-screening is typically done through initial health questionnaires or interviews conducted by clinical coordinators.

Patients are further screened to exclude those with comorbid conditions or contraindications that might interfere with the trial outcomes or pose additional health risks to the participants. This step may require detailed medical evaluations, including a review of the patient's full medical history and possibly additional diagnostic tests to rule out any conditions that would disqualify them from participation.

In one non-limiting example, for diseases like Alzheimer's, certain biomarkers such as amyloid and tau proteins are indicators of disease presence and/or progression. In this step, potential participants may undergo specific biomarker tests, such as cerebrospinal fluid analysis or PET scans, to determine the presence and levels of these biomarkers. Only those whose biomarker levels pass a pre-defined threshold indicative of the disease stage targeted by the clinical trial move forward in the screening process.

Patients who meet all the previous criteria are then formally enrolled in the clinical trial. This involves obtaining informed consent where the participants are made aware of the potential risks and benefits of participating in the trial, the nature of the treatment they will receive, and their rights as participants. Following consent, they are registered as participants, and their treatment regimen begins as per the study protocol.

### Conventional Process

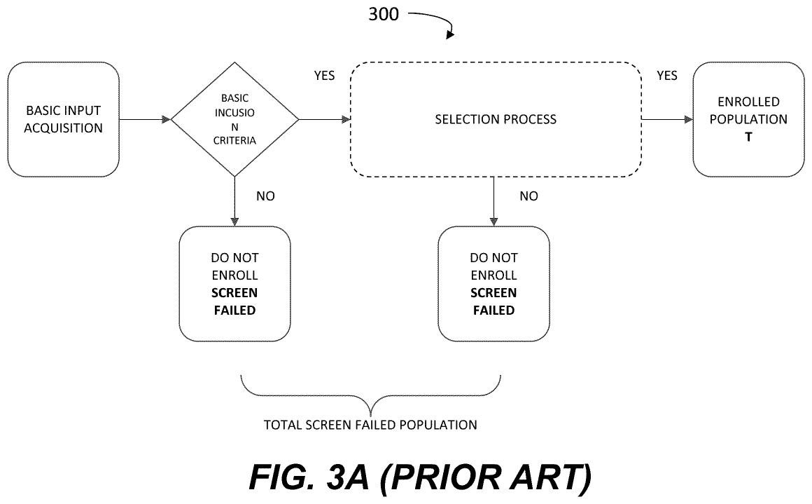

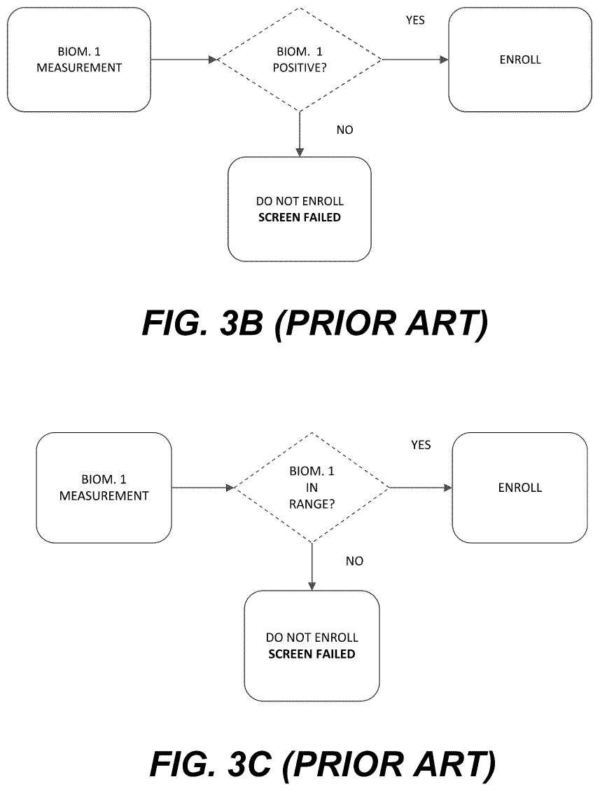

With reference to FIG. 3A, there is depicted a conventional enrollment selection process 300. Input data regarding a candidate population. After verifying basic inclusion and/or exclusion criteria to ensure candidate participants meet the study's requirements. A biomarker-driven selection process is then employed on the remaining candidates. One or more steps involving biomarker analysis is conducted to identify subjects who fall within the desired range and/or cut-off, thereby refining the target population (T). Different scenarios or combination strategies can be performed. With reference to FIGS. 3B and 3C, the conventional process 300 may use a single biomarker being either positive or not, and a single biomarker within a range of acceptable values, respectively.

### Embodiments

Developers of the present technology have devised an enrollment selection process 400 executable by the system server 210, where the biomarker-driven selection process is replaced, at least in part, by a prognostic model 650. In some embodiments, inputs into the prognostic model 650 may at least partially overlap with inputs to the biomarker-driven selection process.

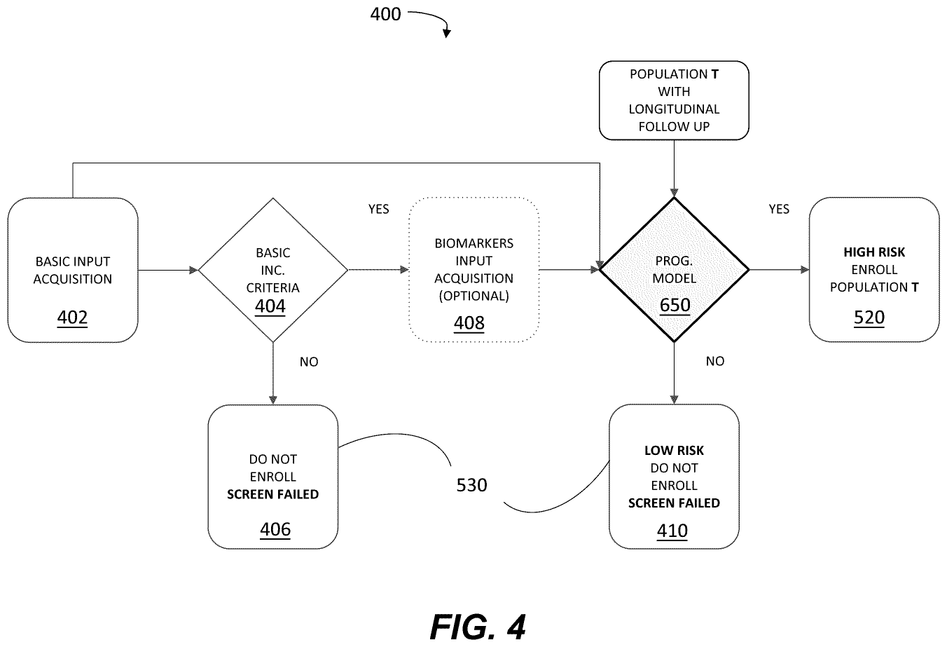

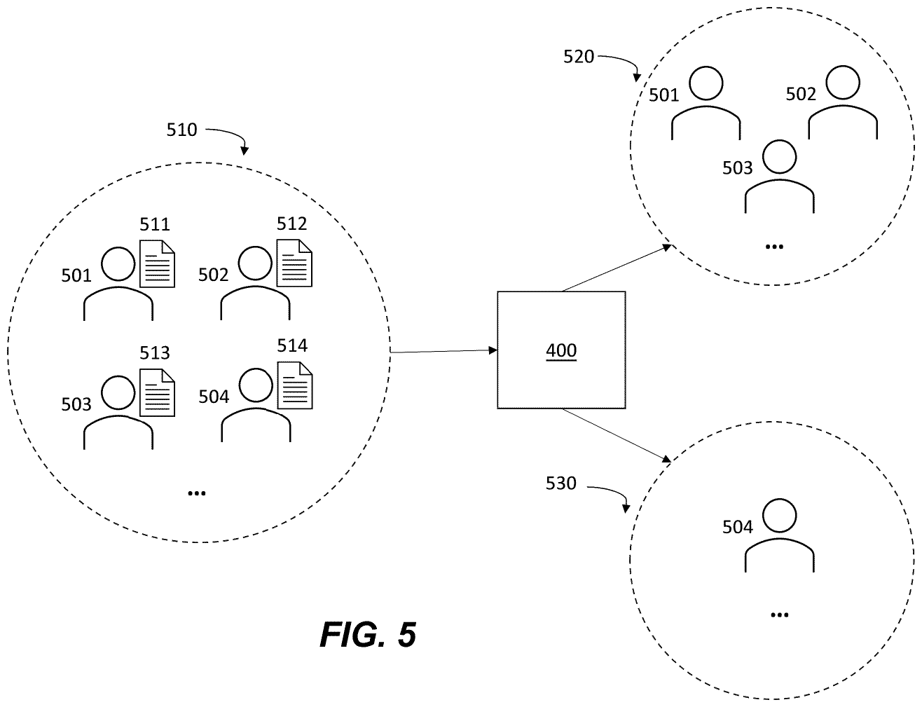

With reference to both FIGS. 4 and 5, More specifically, the system server 210 is configured to acquire input data 402 about a plurality of candidates 510 comprising candidates 501, 502, 503, and 504. For each of the plurality of candidates 510, the system server 210 is configured to acquire candidate-specific data. For example, candidate-specific data 511, 512, 513, and 514 for the candidates 501, 502, 503, and 504, respectively, is acquired by the system server 210 and is used as input into the enrollment selection process 400.

In this embodiment, the system server 210 is configured to perform a first selection process based on pre-determined criteria 404. It is contemplated that the system server 210 may apply one or more heuristic and/or statistical methods for analyzing the candidate-specific data 511, 512, 513, and 514. For example, demographic data about the candidates such as age and cognitive score or clinical stage, for example, may be used for the first selection process.

Based on the first selection process, the system server 210 may be configured to determine a first set of candidates 406 which fail the first selection process and are not enrolled into a target population. The rest of candidates may continue to next steps of the enrollment selection process 400.

Optionally, the system server 210 may be configured to acquire biomarker data 408 about the candidates 501, 502, 503, and 504. In some cases, the biomarker data 408 may be part of the candidate-specific data 511, 512, 513, and 514. In other cases, the biomarker data may be requested by the system server 210 following the first selection process using the pre-determined criteria 404 is completed. In further cases, the enrollment selection process 400 may be executed without the biomarker data 408, without departing from the scope of the present technology.

The system server 210 is configured to input, into the prognostic model 650, the client-specific data about the candidates that remained in the enrollment selection process 400 after the first selection process.

The prognostic model 650 is configured to predict a class value for respective candidates based on the respective client-specific data. In this embodiment, the prognostic model 650 generates a first class value to a set of candidates 520 and a second class value to another set of candidates 410. In this embodiment, the set of candidates 520 is the target population amongst the plurality of candidates 510 and comprises at least the candidates 501, 502, 503. The other set of candidates fail a second selection process and are not enrolled. The set of candidates 406 and the set of candidates 410 form the set of non-enrolled candidates 530 and comprises at least the candidate 504.

In some embodiments of the present technology, the system server 210 may be configured to periodically use the prognostic model 650 on the set of candidates 520. As the clinical trial progresses, periodically updated data about the enrolled individuals may be collected and transmitted to the system server 210. The system server 210 may execute the prognostic model 650 based on the then-current data about the enrolled individuals to determine treatment response.

### Prognostic Model

The prognostic model 650 is a machine learning model configured to predict progression of a medical disease or condition in a candidate. The progression of a medical disease or condition, called an outcome, can be defined as either a change in a continuous variable (e.g. difference between a value at an initial timepoint and the value at a later date) or a discrete variable (e.g. occurrence of a specific event or change in status) over a defined period of time. Examples of a continuous outcome can include, but are not limited to, change in a cognitive score or a change in a biomarker level. Examples of a discrete outcome can include, but are not limited to, a change in diagnosis or change in disease or condition stage.

In some embodiments, a primary outcome may be a continuous variable representing a difference between a value at baseline and a value at follow up. For example, an individual deemed to have progressed will be defined as a patient that displays a change indicative of worsening over a period of time. In the same example, an individual deemed to be stable will be defined as a patient that does not display a change indicative of worsening over a period of time.

As previously alluded to, inputs used to train the prognostic model 650 include candidate-specific information that is typically acquired during the screening/enrollment period of a clinical trial. The inputs may include, but are not limited to: demographic information, clinical assessments (e.g. of either primary or secondary outcomes), imaging, and results of biomarker tests. In at least some embodiments, the prognostic model 650 may be a multimodal model configured to accept input data in a variety of different formats (e.g., text and image).

The system server 210 may be configured to train and test the prognostic model 650 with nested cross-validation techniques, which splits the data into multiple subsets (folds) of separate training and test sets to avoid over-fitting. It is contemplated that samples may be stratified to ensure that every fold maintains the same proportions of target classes that are represented in the overall dataset. In at least some embodiments, the prognostic model 650 may be trained to maximize the Area Under the Receiver Operating Characteristic Curve (ROC AUC).

A variety of architectures may be used for implementing the prognostic model 650. For example, an architecture suitable for predicting either continuous or discrete outcomes can be applied for predicting progression of a medical disease or condition, including, but not limited to: bayesian model, linear and non-linear model, support vector machines, linear regression, random forests, deep learning, neural network, ensembles, etc.

It is contemplated that the prognostic model 650 can provide a continuous prediction of the estimated score or outcome or a classification of one or more states of change from baseline. A continuous variable can also be discretized based on some pre-specified criteria such as, for example: absence of change on the outcome measure through time (suggesting stability), evolution of the outcome measure up to a defined level, or division into quartiles.

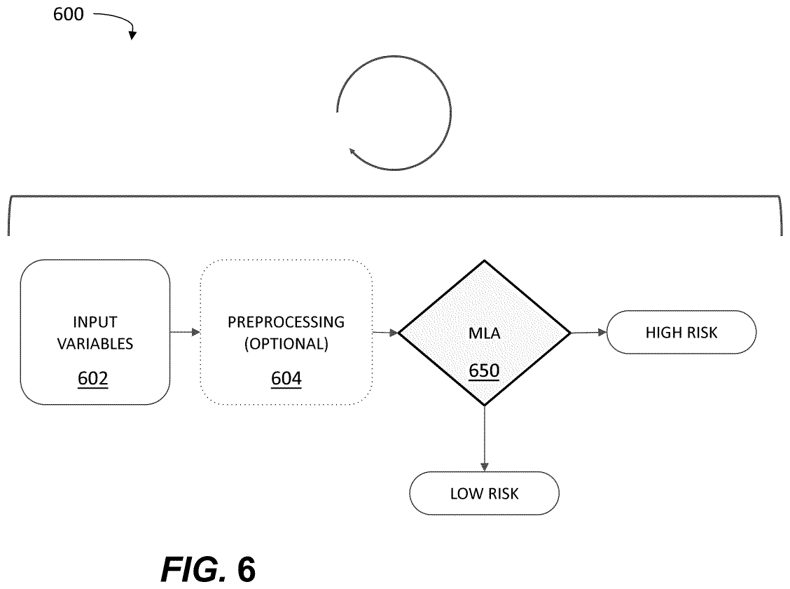

With reference to FIG. 6, there is depicted a training iteration 600 of the prognostic model 650. During the training iteration 600, the system server 210 acquires input variables 602 for a given candidate, the input variables can be optionally preprocessed 604, and are provided as input into the prognostic model 650.

The input variables for the candidate may be associated with a time t. The prognostic model 650 is configured to compute a predicted outcome (binary or continuous) for the candidate at time t+a, where a represents a pre-determined time period. The predicted binary outcome may be compared to a ground-truth binary outcome for the candidate at a moment t+a. The prognostic model 650 may be adjusted based on the comparison as is known in the art.

In at least some embodiment, training the prognostic model 650 to predict progression of a disease or condition may further information about candidate-specific data and outcomes of patients with longitudinal follow-up (e.g. historical data from clinical settings or clinical trials or academic research studies with participants who were observed over time). A training dataset can be formed from this longitudinal data. The training dataset is a set of labeled data (i.e. labeled per patient based on outcomes) that is used to train the prognostic model 650 with a set of features derived from the candidate-specific data including, but not limited to: demographics, medical history, clinical assessments, neuropsychological assessments, cognitive tests, medical images, biomarker results, physiological measures, etc. Cross-validation techniques can be used with the training dataset to avoid over-fitting, tuning hyperparameters, and selecting the best model. With cross-validation, the training dataset is divided into a number of subsets (folds), so that different subsets can be used for training, validation, and testing. It is contemplated that a second independent dataset may be used to assess the predictive performance of the prognostic model 650 in a separate validation process. Metrics for assessing the predictive performance of the prognostic model 650 can include area under the curve (AUC), sensitivity, specificity, positive predictive value (PPV), negative predictive value (NPV), mean absolute error (MAE), mean standard error (MSE), or coefficient of determination (R2), depending on inter alia whether the outcome is a continuous or discrete measure.

### Operating Point Selection Process

In some embodiments of the present technology, the trained model 650 may undergo an operating point selection process for a given clinical trial. The purpose of the operating point selection process of the trained model 650 is to ensure that the trained model 650 outperforms standard biomarker-driven selection processes.

The operating point on a prognostic model's outputs can be tuned to fit specific use cases, such as enriching a clinical trial with a specific population or minimizing a trial's screen failure rate, so that the model may be more or less specific in identifying individuals who will experience disease or condition progression. The goal is to obtain some guarantees of superiority of the selection compared to the reference process. At the upper bound operating point, we obtain equal population quality and sample size requirement/study power while minimizing screen failure rate. At the lower bound operating point, we maximize obtaining a superior quality population with lower sample size requirement/higher study power and same screen failure rate. Any point within that operating range will involve a trade-off between these two extreme properties. How the operation point selection process is performed will now be described in greater detail.

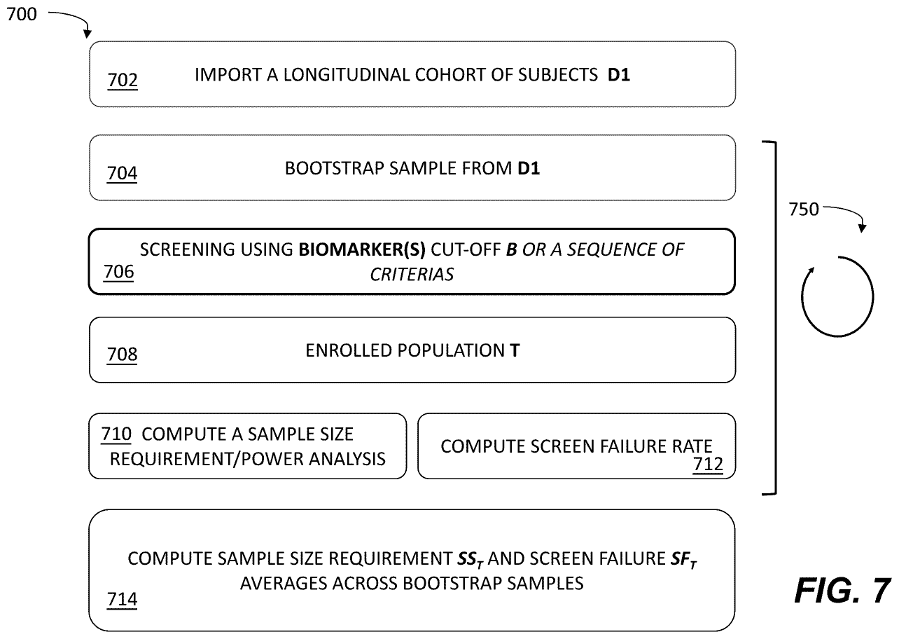

With reference to FIG. 7, there is depicted a scheme-block illustration of a method 700 for determining a reference value for a biomarker-driven process. At step 702, a longitudinal cohort of subjects D1 is provided to the server 210. In this embodiment, D1 is representative of the population getting screened for trial enrollment. At step 704, the server 210 is configured to bootstrap a sample from D1. The bootstrapping step may be employed by the server 210 to estimate variability between samples. From a cohort with longitudinal data, D1, a bootstrap sample can be randomly selected (drawing of sample data with replacement). Other bootstrapping strategies are also contemplated without departing from the scope of the present technology.

At step 706, the server 210 is configured to apply a biomarker-driven selection process or a sequence of criteria. Different strategies can be applied as explained above. For this given bootstrap sample, the server 210 is configured to apply a pre-determined cut-off “B” on a given biomarker and/or a series of screening steps using the given biomarker resulting in the identification of a population “T” that should be recruited into a clinical trial under the standard biomarker process (step 708).

At step 710, the server 210 is configured to compute a sample size requirement and/or a power analysis. For example, if a confidence level of 80% and a particular effect size, is desired, it is possible to compute a sample size estimation to obtain the required sample size required to achieve that statistical power considering the properties of the population and the desired confidence level. At least some techniques used to compute a sample size estimation and obtain the required sample size required to achieve that statistical power considering the properties of the population and the desired confidence level are described in a publication by Schulz, K. F., & Grimes, D. A. (2005), entitled “Sample size calculations in randomised trials: mandatory and mystical” in The Lancet, 365(9467), 1348-1353, and/or in a publication by Noordzij, M., Dekker, F. W., Zoccali, C., & Jager, K. J. (2011), entitled “Sample size calculations”, in Nephron Clinical Practice, 118(4), c319-c323, the contents of which is incorporated herein by reference in its entirety. It is contemplated that a power analysis may be executed to compute a reference sample size requirement for a clinical trial that will recruit a population T. At step 712, the server 210 is configured to compute a screen failure rate. In other words, the server 210 may compute a sub-set of the population from the bootstrap that is to be screened out and the ratio between the number of screened out individuals and the total population being screened is computed

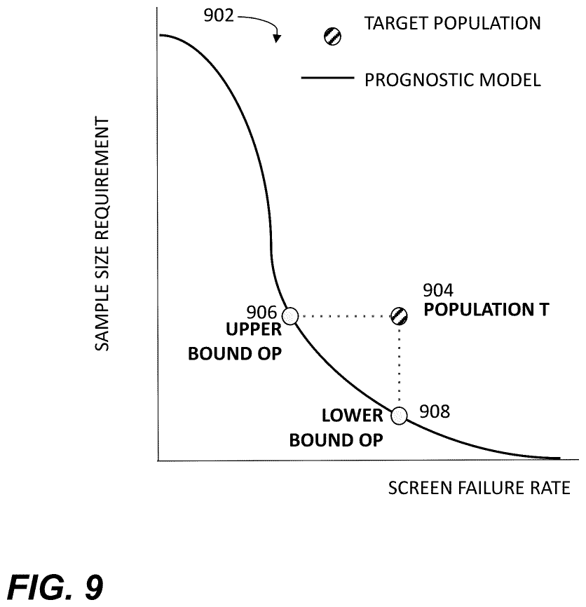

It should be noted that in this embodiment, the server 210 may be configured to execute a number of loops 750. Values for each of the loops 750 can be combined into a combined value for the method 700. For example, the combined value may be an average value, a median value, and the like. At step 714, the server 210 is configured to compute a reference sample size requirement SSₜ and a reference screen failure SFₜ as combined values across the bootstrap samples from the number of loops 750. With reference to FIG. 9, a combined value 904 for population T has the SSₜ value as one coordinate thereof, and SFₜ value as the other coordinate thereof.

In one implementation, the number of loops 750 may be one thousand loops, in order to compute combined values of sample size requirements and screen failure rates across the one thousand bootstrap samples while screening using the biomarker cut-off B and/or a series of biomarker filters with predefined range and/or cut-offs.

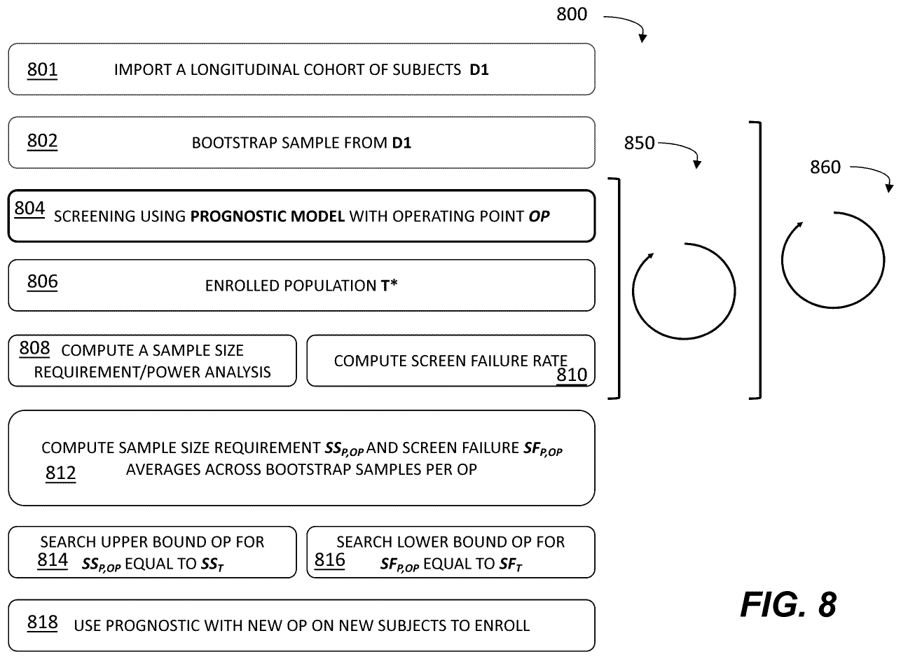

With reference to FIG. 8, there is depicted a scheme-block representation of a method 800 for determining a performance of the prognostic model 650 using a plurality of candidate operating points. As it will be described in detail below, the performance of the prognostic model 650 may be represented via a curve 902, where each point of the curve 902 corresponds to a respective candidate operating point from the plurality of candidate operating points.

At step 801, a population D1 is provided to the server 210. At step 802, the server 210 is configured to execute a bootstrapping operation of the population D1. At step 804, the server 210 may generate, and/or select a candidate operating point from the plurality of candidate operating points, for the prognostic model 650 and perform classification of members of the bootstrap sample. In this embodiment, the candidate operating point may correspond to a classification threshold of the prognostic model 650 for discriminating between a first class and a second class. It is contemplated that candidate operating points may correspond to one or more classification thresholds for the prognostic model 650 for discriminating between a plurality of possible classes that the prognostic model 650 is trained to predict for respective members.

At step 806, the server 210 is configured to determine a population to be enrolled for the bootstrapped sample using the prognostic model 650.

At step 808, the server 210 is configured to compute a sample size requirement and/or execute a power analysis for the bootstrapped sample. At step 810, the server 210 is configured to compute a screen failure rate for the bootstrapped sample.

In this embodiment, the bootstrapped sample is used by server 210 for executing the steps 804, 806, 808, and 810 for each of the plurality of candidate operating points generated and/or selected by the server 210. For example, the server 210 may be configured to execute a number of iterations 850 for a given bootstrapped sample.

In this embodiment, the server 210 may be configured to generate another bootstrapped sample, and execute another number of iterations 860 for the other bootstrapped sample. For example, the server 210 may be configured to execute a plurality of iterations 860 for respective bootstrapped samples generated by the server 210 for subjects D1.

At step 812, the server 210 is configured to compute a given point along the curve 902 by generating a combined value for a given candidate operating point across all bootstrap iterations. To that end, the server 210 may be configured to generate a combined sample size requirement value and a combined screen failure rate for the given candidate operating point across all bootstrap iterations. The combined sample size requirement value and the combined screen failure rate for the given operating point are respective coordinates of a corresponding point along the curve 902.

At step 814, the server 210 is configured to compute an upper operating point 906 by comparing the curve 902 (and/or points thereof) against the combined value 904 computed for the reference biomarker-drive process.

In this embodiment, the upper bound 906 corresponds to a given candidate operating point of the prognostic model 650 which yielded a sample size requirement SSp equal or closest to SSt, while yielding a screen failure rate SFp that is lower than SFt.

It should be noted that for a predetermined cut-off B of a reference biomarker-driven process, the server 210 may determine a prognostic operating point that yields a matching combined sample size requirement on the prognostic split. This operating point on the prognostic output can be an upper bound and may allow for an equivalent quality/power of the selected population as the reference biomarker-driven process but at a comparatively reduced screen failure rate.

At step 816, the server 210 is configured to compute a lower operating point 908 by comparing the curve 902 (and/or points thereof) against the combined value 904 computed for the reference biomarker-drive process.

In this embodiment, the lower bound 908 corresponds to a given candidate operating point of the prognostic model 650 which yielded a screen failure rate SFp equal or closest to SFt, while yielding a sample size requirement SSp that is smaller than SSt.

It should be noted that for the predetermined cut-off B of the reference biomarker-driven process, the server 210 may determine a prognostic operating point that yields a matching combined screen failure rate. This operating point on the prognostic output can be a lower bound and allow for an equivalent screen failure rate to the reference biomarker-driven process but at a comparatively reduced sample size requirement (due to a comparatively better quality of the population).

In some embodiments, the server 210 may be configured to determine a range of target operating points which comprises candidate operating points associated with points of the curve 902 between the upper bound 906 and the lower bound 908. This range of target points between the lower and upper bounds can be applied to the prognostic model 650 to enroll new patients into the clinical trial while allowing for a screen failure rate that will not exceed SFt and/or a sample size estimate that will not exceed SSₜ.

At step 818, the server 210 is used to employ the prognostic model with a target operating point selected between (and including) the upper and lower boundaries 906 and 908 along the curve 902.

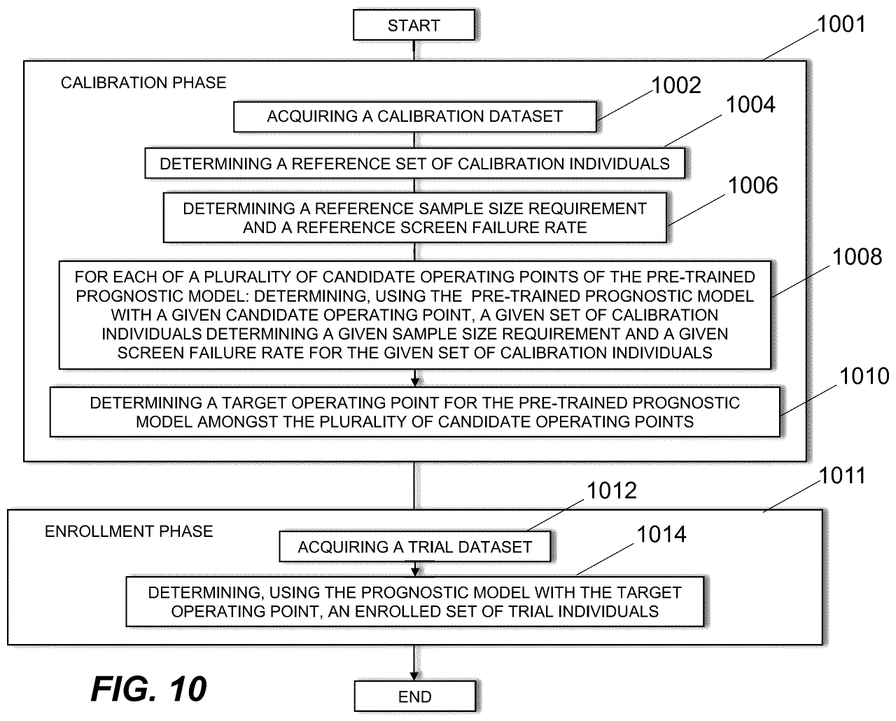

In some embodiments of the present technology, the system server 210 may be configured to execute a method for an enrollment selection process in a clinical trial. With reference to FIG. 10, there is depicted a scheme-block illustration of a method 1000 executable by the system server 210. Various steps of the method 1000 will not be described in greater detail.

It is contemplated that in some embodiments of the present technology, the system server 210 may be configured to execute solely the steps associated with an enrollment phase (e.g., a step 1012 and a step 1014), without departing from the scope of the present technology.

In this embodiment, the server 210 is configured to execute a calibration phase 1001 of a pre-trained prognostic model, such as the prognostic model 650, for example.

At step 1002, the system server 210 may acquire a calibration dataset comprising individual-specific data for a pool of calibration individuals, the pool of calibration individuals matching requirements of the clinical trial.

At step 1004, the system server 210 may determine a reference set of calibration individuals from the pool of calibration individuals that are to be enrolled based on a biomarker-driven selection process applied onto the individual-specific data.

At step 1006, the system server 210 may determine a reference sample size requirement and a reference screen failure rate for the reference set of calibration individuals.

At step 1008, the system server 210 may, for each of a plurality of candidate operating points of the pre-trained prognostic model: determine, using the pre-trained prognostic model with a given candidate operating point, a given set of calibration individuals from the pool of calibration individuals that are to be enrolled based on a model-driven selection process, and determine a given sample size requirement and a given screen failure rate for the given set of calibration individuals determined using the given candidate operation point. The given candidate operating point being a candidate classification threshold for the pre-trained prognostic model for classifying a given calibration individual as being part of an enrolled class and a rejected class.

At step 1010, the system server 210 may determine a target operating point for the pre-trained prognostic model amongst the plurality of candidate operating points based on a comparison between at least one of: the reference sample size requirement and respective sample size requirements of the plurality of candidate operating points; and the reference screen failure rate and respective screen failure rates of the plurality of candidate operating points.

At step 1011, the system server 210 may be configured execute an in-use phase of the prognostic model. During the step 1011, the system server 210 may be configured to execute steps 1012 and 1014.

At step 1012, the system server 210 may acquire a trial dataset comprising individual-specific data for a pool of trial individuals.

At step 1012, the system server 210 may determine, using the prognostic model with the target operating point, an enrolled set of trial individuals from the pool of trial individuals.

In some embodiments, the server 210 may be configured to send data indicative of the selection process to the resource server. It is contemplated that the data generated by the server 210 may be used for administering medication and/or placebo to an enrolled population in accordance with requirements of the clinical trial.

### Case Study

Described here is a non-limiting example of predicting disease or condition progression with a discrete outcome to select patients for a clinical trial.

Placebo groups in Alzheimer's disease (AD) clinical trials often do not show cognitive and functional decline. This lack of clinical progression in participants reduces a trial's statistical power to detect treatment effects and contributes to a trial's failure to meet its clinical endpoints. Clinical trials therefore need strategies to screen out participants who will remain stable in order to enrich participants who will decline within the typical duration of a trial. Trials often use biomarkers of AD pathology to stage individuals and enrich for participants with an expected rate of progression. However, restrictive cutoffs on biomarkers result in high screen failure rates, such that modern AD trials, especially in prodromal stages, fail to enroll over 70% of screened participants. Moreover, since AD pathology exists on a continuum, binary cutoffs can exclude individuals who have levels of pathology just beyond the biomarker cutoffs but who still have the desired clinical progression. A prognostic model that predicts individual-level disease progression can be useful for identifying participants who will have a specific clinical trajectory while minimizing screen failure rates due to biomarker cutoffs. In this study, a prognostic machine learning model was trained according to certain implementations of the present technology, to identify patients with early AD who would experience decline on a common trial endpoint, the Clinical Dementia Rating-Sum of Boxes (CDR-SB) from those who would remain stable on the CDR-SB over a two year period. Individuals who are predicted to decline by the trained model are then recommended for inclusion into a clinical trial. We compared the screen failure rates and sample size estimates associated with selecting participants for a hypothetical trial based on an AD biomarker cutoff, phosphorylated tau 181 (ptau181) levels in the cerebrospinal fluid, and the predictions from the trained model.

A discovery dataset of 985 participants with early AD who had 2282 visits was compiled (Cohort A). Cohort A was used for training the prognostic model. A second independent dataset of 164 participants with early AD who had 264 visits was compiled (Cohort B). Cohort B was used for external validation. We included data from participants only if they met the following criteria: 1) aged between 50-90 years old, 2) CDR global scores between 0.5-1, 3) had evidence of amyloid pathology, 4) were not depressed as measured by a Geriatric Depression Scale score of 5 or less, 5) had no known history of schizophrenia and/or bipolar disorder, 6) were not taking anti-anxiety medications, antipsychotic agents, or medications for Parkinson's disease, and 7) had at least 2 years of follow-up.

The prognostic model was trained in ADNI to classify individuals as likely decliners, if the change on CDR-SB was predicted to be at least +0.5 points in 2 years, or likely stable patients if the change on CDR-SB was predicted to be 0 points or less (i.e. negative change) in 2 years. The CDR-SB ranges from 0 to 18, where higher scores indicate worse impairment, so a negative change indicates improvement and a positive change indicates worsening. The following features were included as inputs in the model: baseline CDR-SB score, baseline Mini-Mental State Examination (MMSE) score, baseline cerebrospinal fluid (CSF) measurements of amyloid beta 42, ptau181, and total tau, baseline age, sex, and number of APOE4 alleles. Stratified five-fold nested cross-validation was done to tune the hyperparameters. AD-Px achieved an area under the curve (AUC) of 80.1% (95% CI [77.1, 83.0%]) to predict likely decliners, while cutoffs on CSF ptau181 achieved an AUC of 71.6% [68.1, 74.8%]. That trained model achieved a higher AUC than CSF ptau181 indicates that the trained model has superior prognostic performance.

We bootstrapped 1000 samples from the Cohort A. We found that applying a single cutoff on CSF ptau181, at a level that can separate patients with AD dementia from cognitively unimpaired individuals, screen failed 36.2% (95% CI [32.0, 40.5%]) of participants, on average across the bootstrapped samples. Power analyses estimated that a total of 453 [366, 544] individuals, on average, would be required for a two-arm clinical trial to detect a 30% treatment effect with 80% power if the trial exclusively enrolled participants who met the biomarker cutoff. For each bootstrapped sample, we also estimated screen failure rates and sample size estimates for all possible operating points of the trained model for a hypothetical clinical trial that would selectively enroll participants if they were predicted to be likely decliners. At an operating point on the trained model that matched the sample size estimate required by the CSF ptau181 biomarker (AD-Px N=453 [382, 531]), the trained model screen failed 16.7% [13.9, 20.1%] of participants. This first operating point demonstrates that the trained model can enrich a clinical trial with equivalent statistical power as a biomarker with a significantly lower screen failure rate. At a second operating point on the trained model that matched the screen failure rate of the CSF ptau181 cutoff, the trained model required a smaller total sample size of 362 [298, 428]. This second operating point demonstrates that the trained model can be used to enrich a trial above the enrichment capability of a biomarker, thus requiring a smaller sample size, while maintaining an equivalent screen failure rate.

The results were replicated on the external validation cohort (Cohort B). In 1000 bootstrapped samples of the Cohort B, the CSF ptau181 cutoff screen failed 42.1% [36.0, 48.2%] of participants, while requiring a sample size of 518 [379, 700] to adequately power a clinical trial. Applying the matched sample size operating point derived in Cohort A on the trained model predictions in Cohort B yielded a similar sample size estimate of 508 [379, 663] participants as the CSF ptau181 cutoff, but the trained model screen failed a smaller percentage of participants at a rate of 11.6% [7.3, 15.8%]. Then, at an operating point that matched the biomarker's screen failure rate of 42%, power analyses estimated that the trained model required fewer participants with a total N=369 [269, 503] compared to the biomarker.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A method for determining an operating point for a trained model, wherein the trained model was trained to predict a likelihood of progression of a disease or a condition, the method comprising:
acquiring a calibration dataset comprising individual-specific data for a pool of calibration individuals;
selecting, based on a biomarker, a set of calibration individuals from the pool of calibration individuals;
determining a reference sample size requirement and a reference screen failure rate for the set of calibration individuals;
for each of a plurality of candidate operating points of the trained model:
determining, using the trained model with a given candidate operating point, a given set of calibration individuals from the pool of calibration individuals that are to be enrolled based on a model-driven selection process,
the given candidate operating point being a candidate classification threshold for the trained model for classifying a given calibration individual as being part of an enrolled class or a rejected class; and
determining a given sample size requirement and a given screen failure rate for the given set of calibration individuals determined using the given candidate operating point; and
selecting the operating point for the trained model amongst the plurality of candidate operating points based on a comparison between at least one of:
the reference sample size requirement and respective sample size requirements of the plurality of candidate operating points; and
the reference screen failure rate and respective screen failure rates of the plurality of candidate operating points.

2. The method of claim 1, wherein each individual of the pool of calibration individuals satisfies requirements of a clinical trial.

3. The method of claim 2, wherein the operating point is an upper target operating point determined based on a comparison of the reference sample size requirement and the respective sample size requirements of the plurality of candidate operating points, wherein the upper target operating point is configured for reducing a screen failure rate of individuals for the clinical trial.

4. The method of claim 2, wherein the operating point is a lower target operating point determined based on a comparison of the reference screen failure rate and the respective screen failure rates of the plurality of candidate operating points, wherein the lower target operating point is configured for reducing a sample size of individuals for the clinical trial.

5. The method of claim 1, wherein the operating point comprises a range of target operating points determined based on both (i) the reference sample size requirement and the respective sample size requirements of the plurality of candidate operating points, and (ii) the reference screen failure rate and the respective screen failure rates of the plurality of candidate operating points.

6. The method of claim 1, further comprising:
acquiring a trial dataset comprising individual-specific data for a pool of trial individuals; and
determining, using the trained model and the operating point, a set of individuals to enroll in A clinical trial from the pool of trial individuals.

7. A method for training a model to forecast a likelihood of progression of a disease or a condition after a pre-determined interval, the method comprising:
receiving a training dataset comprising a plurality of training datapoints, wherein each training datapoint comprises data corresponding to a patient and a label indicating a ground truth outcome for the patient;
training the model to predict, for each training datapoint of the plurality of training datapoints, and based on the data corresponding to a respective training datapoint, a predicted outcome for the patient associated with the respective training datapoint;
comparing, for each training datapoint of the plurality of training datapoints, the predicted outcome for a respective training datapoint to the ground truth outcome for the respective training datapoint; and
adjusting, based on the comparing, the model.

8. The method of claim 7, further comprising determining an operating point for the model, wherein determining the operating point comprises:
acquiring a calibration dataset comprising individual-specific data for a pool of calibration individuals;
selecting, based on a biomarker, a set of calibration individuals from the pool of calibration individuals;
determining a reference sample size requirement and a reference screen failure rate for the set of calibration individuals;
for each of a plurality of candidate operating points of the model:
determining, using the model with a given candidate operating point, a given set of calibration individuals from the pool of calibration individuals that are to be enrolled based on a model-driven selection process,
the given candidate operating point being a candidate classification threshold for the model for classifying a given calibration individual as being part of an enrolled class or a rejected class; and
determining a given sample size requirement and a given screen failure rate for the given set of calibration individuals determined using the given candidate operating point; and
selecting the operating point for the model amongst the plurality of candidate operating points based on a comparison between at least one of:
the reference sample size requirement and respective sample size requirements of the plurality of candidate operating points; and
the reference screen failure rate and respective screen failure rates of the plurality of candidate operating points.

9. The method of claim 8, further comprising:
acquiring a trial dataset comprising individual-specific data for a pool of trial individuals; and
determining, using the model and the operating point, a set of individuals to enroll in A clinical trial from the pool of trial individuals.

10. A method for an enrollment selection process in a clinical trial, the method comprising:
acquiring a trial dataset comprising individual-specific data for a pool of trial individuals; and
determining, using a pre-trained model an enrollable set of trial individuals from the pool of trial individuals, wherein:
the pre-trained model is a prognostic model that has been trained to forecast a likelihood of progression of a disease or a condition after a pre-determined interval; and
the pre-trained model has a dynamic operating point.

11. The method of claim 10, wherein the pre-trained model is configured to reduce a sample size requirement of the enrollable set of trial individuals in comparison to a sample size requirement of the enrollable set of trial individuals when a solely biomarker-driven process is used to determine the enrollable set of trial individuals.

12. The method of claim 10, wherein the pre-trained model is configured to reduce a screen failure rate of the enrollable set of trial individuals in comparison to a screen failure rate of the enrollable set of trial individuals when a solely biomarker-driven process is used to determine the enrollable set of trial individuals.

13. The method of claim 10, wherein the dynamic operating point is a target operating point selected from a plurality of candidate operating points, the selection of the target operating point being based on at least one of (i) a sample size requirement for the clinical trial and (ii) a screen failure rate for the clinical trial.

14. The method of claim 10, wherein the method further comprises executing a calibration phase of the pre-trained model for determining a target operating point amongst a plurality of candidate operating points, and wherein executing the calibration phase comprises:
acquiring a calibration dataset comprising individual-specific data for a pool of calibration individuals, the pool of calibration individuals matching requirements of the clinical trial;
determining a reference set of calibration individuals from the pool of calibration individuals that are to be enrolled based on a biomarker-driven selection process applied onto the individual-specific data;
determining a reference sample size requirement and a reference screen failure rate for the reference set of calibration individuals;
for each of the plurality of candidate operating points of the pre-trained model:
determining, using the pre-trained model with a given candidate operating point, a given set of calibration individuals from the pool of calibration individuals that are to be enrolled based on a model-driven selection process,
the given candidate operating point being a candidate classification threshold for the pre-trained model for classifying a given calibration individual as being part of an enrolled class and a rejected class; and
determining a given sample size requirement and a given screen failure rate for the given set of calibration individuals determined using the given candidate operating point; and
determining the target operating point for the pre-trained model amongst the plurality of candidate operating points based on a comparison between at least one of:
(i) the reference sample size requirement and respective sample size requirements of the plurality of candidate operating points; and
(ii) the reference screen failure rate and respective screen failure rates of the plurality of candidate operating points.

15. The method of claim 14, wherein the target operating point is an upper target operating point determined based on a comparison of the reference sample size requirement and the respective sample size requirements of the plurality of candidate operating points, the upper target operating point for reducing a screen failure rate of an enrolled set of trial individuals for a same sample size requirement of the enrolled set of trial individuals if a biomarker-driven process has been used to determine the enrolled set of trial individuals.

16. The method of claim 14, wherein the target operating point is a lower target operating point determined based on a comparison of the reference screen failure rate and the respective screen failure rates of the plurality of candidate operating points, the lower target operating point for reducing a sample size requirement of an enrolled set of trial individuals for a same screen failure rate of the enrolled set of trial individuals if a biomarker-driven process has been used to determine the enrolled set of trial individuals.

17. The method of claim 14, wherein the target operating point is a range of target operating points determined based on both (i) the reference sample size requirement and the respective sample size requirements of the plurality of candidate operating points, and (ii) the reference screen failure rate and the respective screen failure rates of the plurality of candidate operating points, and wherein the method further comprises determining an enrolled set of trial individuals using the pre-trained model with an operating point within the range of target operating points.

18. The method of claim 14, wherein the individual-specific data comprises multimodal data.

19. The method of claim 14, wherein the individual-specific data comprises biomarker data.

20. The method of any one of claim 10, wherein the pre-trained model is at least one of: a Bayesian model, a support vector machine, a linear regression model, a random forest model, a deep learning model, an ensemble-based model.
